(19) United States
(12) Patent Application Publication
(10) Pub. No.: US 2006/0144869 A1
(43) Pub. Date: Jul. 6, 2006

(54) Container closure delivery system

(76) Inventors: Byeong Seon Chang, Thousand Oaks, CA (US); Roger Wongjung Liu, Moorpark, CA (US)

Correspondence Address: Craig A. Crandall, APC, 3034 Deer Valley Avenue, Newbury Park, CA, 91320, US

(21) Appl. No.: 11/172,064

(22) Filed: Jun. 30, 2005

Related U.S. Application Data
(60) Provisional application No. 60/640,625, filed on Dec. 30, 2004.

(51) Int. Cl. G01F 11/00; B67D 5/42

(52) U.S. Cl. 222/386

(57) ABSTRACT

The present invention relates to a container closure delivery system that is suitable for lyophilized pharmaceutical injectable powder products. The system comprises storage stable powder formulations and a container closure assembly design wherein the formulation can be filled and lyophilized with a standard fill finish equipment, and the formulations and lyophilization processes are optimized to produce a powder that readily dissolves upon contact with a diluent, thereby facilitating the direct injection of the lyophilized product without the need for a separate reconstitution/mixing/priming step.

This application claims the benefit of U.S. Provisional Application No. 60/640,625, filed Dec. 30, 2004.

### TECHNICAL FIELD

The field of the present invention is a container closure delivery system that is suitable for lyophilized pharmaceutical injectable products and which facilitates the easy, direct injection of the lyophilized product without the need for a reconstitution/mixing step of the powder and a liquid diluent.

### BACKGROUND OF THE INVENTION

Due to continued advances in genetic and cell engineering technologies, proteins known to exhibit various pharmacological actions in vivo are capable of production in large amounts for pharmaceutical applications. However, one of the most challenging tasks in the development of protein pharmaceuticals is to deal with the inherent physical and chemical instabilities of such proteins, especially in aqueous dosage forms. To try to understand and maximize the stability of protein pharmaceuticals and any other usable proteins, many studies have been conducted, especially in the past two decades. These studies have covered many areas, including protein folding and unfolding/denaturation, mechanisms of chemical and physical instabilities of proteins, as well as various means of stabilizing proteins in aqueous form; see, e.g., Manning et al., Pharm Res., 1989;6:903-918; Arakawa et al., Adv Drug Deliv Rev., 2001;46:307-326; Wang W., Int J Pharm., 1999;185:129-188; Chen T., Drug Dev Ind Pharm., 1992;18:1311-1354, and references cited therein.

Because of the instability issues associated with the aqueous dosage forms, powder formulations are generally preferred to achieve sufficient stability for the desired shelf life of the product. Various techniques to prepare dry powders have been known, substantiated and practiced in the pharmaceutical and biotech industry. Such techniques include lyophilization, spray-dying, spray-freeze drying, bulk crystallization, vacuum drying, and foam drying. Lyophilization (freeze-drying) is often a preferred method used to prepare dry powders (lyophilizates) containing proteins. Various methods of lyophilization are well known to those skilled in the art; see, e.g., Pikal M J., In: Cleland J L, Langer R. eds. Formulation and Delivery of Proteins and Peptides. Washington, D.C.: American Chemical Society; 1994:120-133; Wang W., Int J Pharm. 2000;203:1-60, and references cited therein. The lyophilization process consists if three stages: freezing, primary drying, and secondary drying. Because the protein product is maintained frozen throughout drying process, lyophilization provides the following advantages over alternative techniques: minimum damage and loss of activity in delicate, heat-liable materials; speed and completeness of rehydration; the possibility of accurate, clean dosing into final product containers so that particulate and bacterial contamination is reduced; permits product reconstitution at a higher concentration than it was at the time of freezing; and permits storage of the product at ambient temperatures. The latter can be particularly useful for hospital products in areas that do not have ready access to freezers, especially ultra-cold freezers.

Unfortunately, even in solid dosage forms, some proteins can be relatively unstable and this instability may be a product of the lyophilization method used for preparing the solid dosage forms and/or the inherent instability of the actual solid dosage formulations themselves. For example, in certain instances, lyophilization processing events can force a protein to undergo significant chemical and physical changes. Such processing events include concentration of salts, precipitation, crystallization, chemical reactions, shear, pH, amount of residual moisture remaining after freezedrying, and the like. Such chemical and physical changes include, e.g., formation of dimer or other higher order aggregates, and unfolding of tertiary structure. Unfortunately, these changes may result in loss of activity of the protein, or may result in significant portions of the active materials in the drug having been chemically transformed into a degradation product or products which may actually comprise an antagonist for the drug or which may give rise to adverse side effects. In addition to the instabilities incurred upon proteins because of the inherent steps of the lyophilization process, other disadvantages of lyophilization include: long and complex processing times; high energy costs; and expensive set up and maintenance of the lyophilization facilities. As such, use of lyophilization is usually restricted to delicate, heat-sensitive materials of high value. Additionally, lyophilized powders are typically formed as cakes, which require additional grinding and milling and optionally sieving processing steps to provide flowing powders. To try to understand and to optimize protein stability during lyophilization and after lyophilization, many studies have been conducted; see, e.g., Gomez G. et al., Pharm Res. 2001;18:90-97; Strambini G B., Gabellieri E., Biophys J., 1996;70:971-976; Chang B S. et al., J Pharm Sci., 1996;85:1325-1330, Pikal M J., Biopharm, 1990;3:9, Izutsu K. et al., Pharm. Res., 1994;11-995, Overcashier D E., J Pharm Sci., 1999;88:688, Schmidt E A. et al., J Pharm Sci., 1999;88:291, and references cited therein.

In order to allow for parenteral administration of these powdered drugs, the drugs must first be placed in liquid form. To this end, the drugs are mixed or reconstituted with a diluent before being delivered parenterally to a patient. The reconstitution procedure must be performed under sterile conditions, and in some procedures for reconstituting, maintaining sterile conditions is difficult. One way of reconstituting a powdered drug is to inject a liquid diluent directly into a drug vial containing the powdered drug. This can be performed by use of a combination-syringe and syringe needle having diluent contained therein and drug vials which include a pierceable rubber stopper. The method of administration goes as follows: 1) the rubber stopper of the drug vial is pierced by the needle and the liquid in the syringe injected into the vial; 2) the vial is shaken to mix the powdered drug with the liquid; 3) after the liquid and drug are thoroughly mixed, a measured amount of the reconstituted drug is then drawn into the syringe; 4) the syringe is then withdrawn from the vial and the drug then be injected into the patient.

Other methods of administration of powdered drugs include the use of dual-chambered injection cartridges and/or pre-filled syringe systems. Injection cartridges of the dual-chamber type are well-known and have found a wide use. They are used together with various types of injection apparatuses which serve to hold the cartridge as it is readied for injection and as injections are subsequently administered. Injection cartridges of the dual-chamber type generally comprise a cylindrical barrel, which is shaped like a bottleneck at its front end and has an open rear end. The front end is closed by a septum of rubber or other suitable material, which is secured in place by means of a capsule. This capsule has a central opening where the septum is exposed and may be pierced by a hollow needle to establish a connection with the interior of the cartridge; see e.g., U.S. Pat. No. 5,435,076 and references cited therein.

Dual-chambered pre-filled syringe systems are well known and have found wide commercial use; see e.g., U.S. Pat. Nos. 5.080,649; 5,833,653; 6,419,656; 5,817,056; 5,489,266, and references cited therein. Pre-filled syringes of the dual-chambered type generally comprise an active ingredient which is lyophilized in one chamber, while a second chamber of the syringe contains a solvent that is mixed with the active substance immediately before application. In such devices, in order to facilitate the movement of the syringe plunger against compression of air, the chamber containing the lyophilized product typically has large head space and some additional mechanism, e.g., rotation of the plunger, screwing in the plunger, is necessary. As a result, the reconstituted drug needs to primed to remove large volumes of air prior to injection; see e.g., U.S. Pat. No. 6,817,987 which describes a hypodermic syringe which holds a solvent and a soluble component (medicament) and wherein the solvent and medicament are mixed as the user presses and then releases the plunger of the syringe. Upon complete mixing, the user attaches a needle and then rotates the plunger of the syringe to allow for the injection.

Other devices used for reconstitution and delivery of powdered drugs are described in, e.g., U.S. Pat. Nos. 4,328,802; 4,410,321; 4,411,662; 4,432,755; 4,458,733; 4,898,209; 4,872,867; 3,826,260, and references cited therein. Unfortunately, all of these known methods require thorough reconstitution/mixing/priming of the lyophilized product into the diluent prior to injection and this reconstitution step can be complex, arduous and tedious for the patient. The need for this additional reconstitution/mixing/priming step renders injection of lyophilized product with convenient delivery devices such as autoinjectors unfeasible. On the contrary, liquid formulations do not require such preparation and can be delivered with convenient prefilled syringes and/or autoinjectors.

While these studies and advances have furthered the technology, there still clearly exists a need for improved storage stable powder drug formulations and improved lyophilization processes which are less complex and more economical, which do not lead to protein instability during processing, and which produce stable protein powders (at room temperature) for the desired shelf life of the product. There also still clearly exists a need for improved methods for the delivery of powdered drugs which do not require a reconstitution/mixing/priming step of the powdered drug with a diluent.

### SUMMARY OF THE INVENTION

The present invention provides for a container closure delivery system that is suitable for lyophilized pharmaceutical injectable products and facilitates the easy, direct injection of the lyophilized product without the need for a reconstitution/mixing step of the powder and a liquid diluent. The present invention utilizes powder formulations and lyophilization processes that are optimized to produce powders which provide for “rapid” dissolution of the lyophilized powder, i.e., the powders are readily and immediately dissolved upon contact with a liquid diluent.

One object of the present invention is to provide a new container closure assembly suitable for lyophilized pharmaceutical injectable products and designed to provide for direct injection of a lyophilized product without the need for a reconstitution/mixing/priming step of the powder and diluent prior to injection. The container closure assembly of the present invention consists of three operating components designed to function in a manufacturing function and an end user function: a product container component; a soft plug component; and a luer slip/luer lock hard plug component. The container closure assembly is specifically designed to have minimal head space to avoid the need for priming. The product container component is specifically designed to hold a liquid to be lyophilized and capable of holding a plunger assembly. The soft plug component and hard plug component are specifically designed to engage with each other to form a plunger assembly with can then be inserted into the product container. Upon completion of the lyophilization process, the plunger assembly is compressed such that it rests directly on top of the powdered pharmaceutical product, i.e., there is no air space between the powder and the plunger assembly, and the plunger assembly serves as a one way valve to allow for the flow of liquid into the container closure assembly, i.e., allow for liquid to encounter the powder and rapidly reconstitute. Importantly, the container closure assembly is designed to utilize or be easily adaptable to industry standard or existing filling systems, providing a more economical alternative. Because of the unique assembly design, the container closure assembly facilitates the easy, direct injection of the lyophilized product without the need for a reconstitution/mixing/priming step of the powder and a liquid diluent by the end user.

Another object of the present invention is an improved process for the preparation of a container closure assembly containing a lyophilized powder product. This improved process comprises the following steps: 1) utilizing a industry standard vial manufacturing filling line, the product container is loaded into the equipment in a similar manner as regular vials; 2) the product container is filled with liquid active ingredient; 3) the hard plug portion is inserted snugly into the soft plug portion to create a plunger assembly; 4) the plunger assembly is dropped into an “open” position on top of the product container, sealing the product container in the same manner as lyophilization stoppers are mounted to regular vials; 5) the complete container closure assembly is then placed into the lyophilizer; 6) upon lyophilization, vapor is allowed to escape via the openings within the plunger assembly; and 7) upon completion of lyophilization, vertical compression of the lyophilizer shelves will seal the plunger assembly into the product container creating a sealed container closure assembly which retains the sterility of the active ingredient.

Another object of the present invention is an improved method for the administration of a lyophilized pharmaceutical powder product using the container closure system of the present invention. This improved method of administration comprises the following steps: 1) the sealed container closure assembly containing the lyophilized powder product with minimal head space is attached at one end via friction fit to either a luer-lock or luer-slip syringe containing the diluent; 2) a tangential force is applied to the detachable base at the end of the neck area of the container closure assembly, thus breaking off the base and exposing a tip for the attachment of a standard type needle; 3) a standard type needle is attached to said exposed tip of the container closure assembly; 4) the injection is then initiated as normal by inserting the needle into the injection site; and 5) force is applied to the syringe plunger whereupon the diluent in the syringe will be forced through the container closure assembly, encounter the lyophilized powder and rapidly reconstitute the powder to allow the liquefied product mixture to flow into the injection site, completing the injection. Importantly, there is no requirement for a reconstitution/mixing/priming step of the powder and diluent by the end user.

### BRIEF DESCRIPTION OF THE FIGURES

FIG. 1 shows a cross-sectional view of the product container component of the container closure assembly of the present invention.

FIG. 2 shows an isometric view of the soft plug portion of the container closure assembly of the present invention.

FIG. 3 shows a cross sectional view of the soft plug portion of the container closure assembly of the present invention.

FIG. 4 shows a cross sectional view of the hard plug portion of the container closure assembly of the present invention.

FIG. 5 shows a cross sectional view of an embodiment of the container closure assembly whereupon a plunger assembly consisting of a soft plug portion and a hard plug portion are installed upon the product container after the filling the product container with liquid active ingredient and prior to placement of the container closure assembly within a freeze drying apparatus, i.e., the plunger assembly is installed in an “open” position in the product container.

FIG. 6 shows a cross sectional area of an embodiment of the container closure assembly upon completion of the freeze drying cycle whereupon the liquid active ingredient has formed into a dry powder and the plunger assembly has been compressed by the freeze dryer shelves to create a sealed container closure assembly.

FIG. 7 shows the intended use of the sealed container closure assembly of the present invention with a pre-filled syringe and needle. In FIG. 7, the base attached to the neck area of the assembly has been broken off to allow for attachment of a needle.

FIG. 8 is a graph depicting the ‘gradient delivery’ injection profile associated with the administration of a powdered drug using the powder formulations, lyophilization processes, and container closure assembly of the present invention. Protein concentration is plotted versus cumulative injection volume.

FIG. 9 is a graph depicting an injection profile representative of those associated with the administration of powdered drugs using prior art devices which require a reconstitution and/or mixing step of the powdered drug with a diluent prior to injection. Protein concentration is plotted versus cumulative injection volume.

### DETAILED DESCRIPTION OF THE INVENTION

As those in the art will appreciate, the foregoing detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

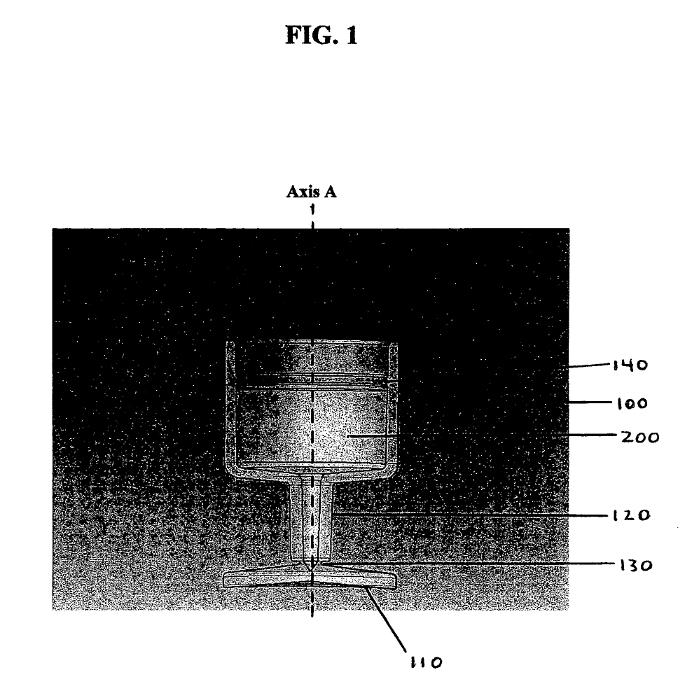

Referring now in more detail to the drawings, FIG. 1 shows the product container 100 (also referred to herein as Component C) of the described container closure assembly 600. The product container 100, whose vertical axis is described by axis A, is constructed of a suitable plastic material, is cylindrical in shape, and has at one end an opening and at the other end an ejection port with detachable base 110. The circular radius of the product container 100 wall creates a sufficient holding volume of liquid active ingredient 200. Moving down the vertical axis A, the radius of the container reduces to form the neck area 120 of the product container. The outer surface area of this neck area 120 is of a sufficient radius to allow for a friction fit of a standard type luer slip or luer lock syringe needle attachment. At the end of the neck area 120, a break or scoring point 130 is formed such that when the base 110 is torqued, it will break off at this point 130. The base 110 is of a circular shape and designed to be grasped and torqued and removed when forces are presented in any matter other than vertically, along axis A. A locking ridge 140 is integrated into the sidewall of the product container 100 such that upon full insertion of the plunger assembly 500, the plunger assembly 500 cannot be removed.

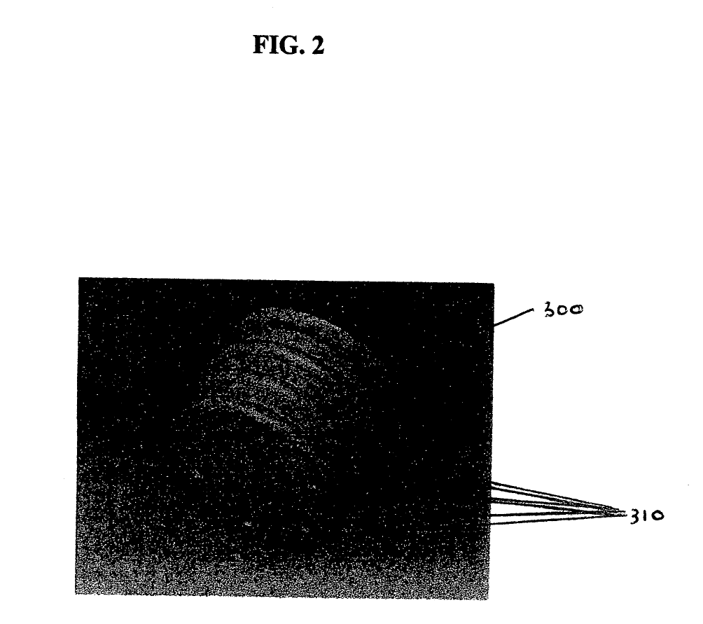

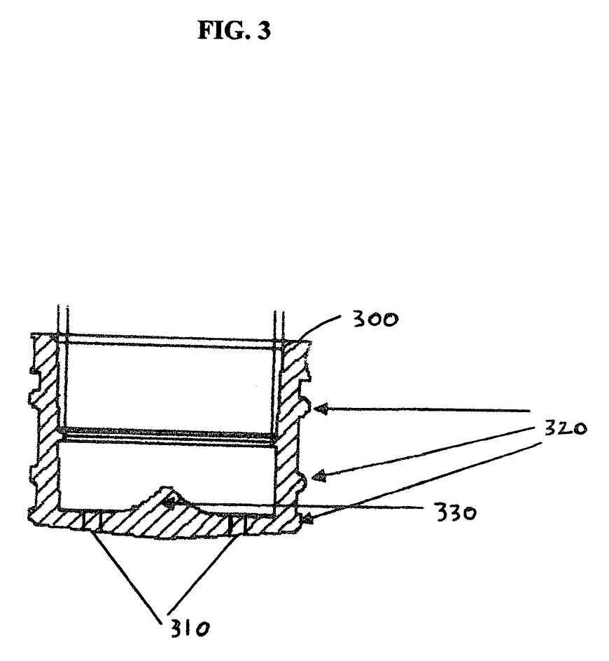

FIGS. 2 and 3 show the soft plug portion 300 (also referred to herein as Component B) of the described container closure assembly 600. This soft plug portion 300 is envisaged to be constructed out of a suitable material that can offer appropriate sealing properties. The soft plug portion 300 has a hollow inside and is constructed to accept the hard plug portion 400 to create a plunger assembly 500 for the container closure assembly 600. In FIG. 2, vent holes 310 are depicted which allow for vapors to escape during lyophilization processes. In FIG. 3, sealing ridges 320 are depicted which serve to seal the soft plug portion 300 against the interior wall of the product container 100. Also depicted in FIG. 3 is a sealing conical mound 330 which serves to seal the active ingredient during manufacturing and which is the male portion that when mated with the depression 430 of the hard plug portion 400 in the sealed container closure assembly 600, will form a one way valve during patient use.

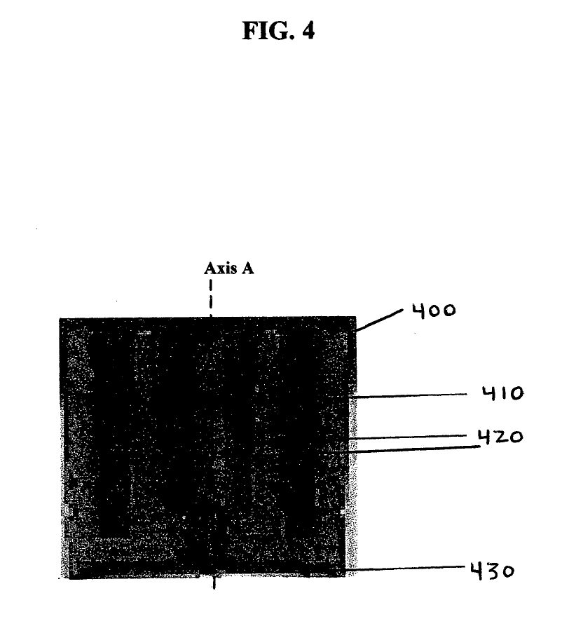

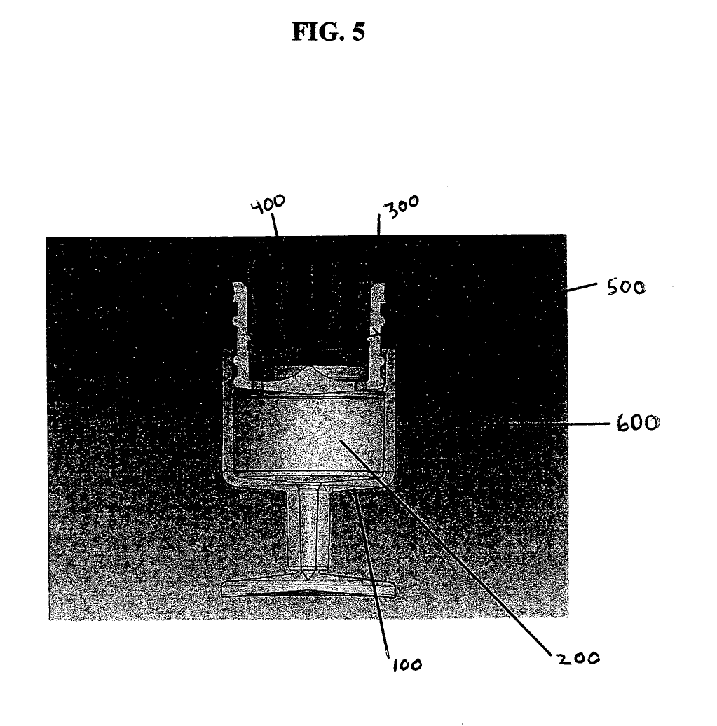

FIG. 4 shows the luer slip/luer lock hard plug portion 400 (also referred to herein as Component A) of the described container closure assembly 600. This hard plug portion 400, whose vertical axis is described by axis A, is envisaged to be constructed of a suitable plastic material. In FIG. 4, a female luer slip fitting cavity 410 is depicted where a standard type luer slip syringe can be frictionally attached. Also in FIG. 4, a circular cavity 420 is depicted (when viewed down upon axis A) that can accommodate a typical luer lock fitting found on most existing luer lock syringes. Also depicted in FIG. 4 is a depression 430 and female portion that when mated with the sealing conical mound 330 of soft plug portion 300 in the sealed container closure assembly 600, will form a one way valve during patient use. FIG. 5 shows a cross sectional view of an embodiment of the container closure assembly 600 whereupon a plunger assembly 500 consisting of a soft plug portion 300 and a hard plug portion 400 are installed upon the product container 100 after the filling the product container 100 with liquid active ingredient 200 and prior to placement of the container closure assembly within a freeze drying apparatus, i.e., the plunger assembly 500 is installed in an “open” position in the product container 100.

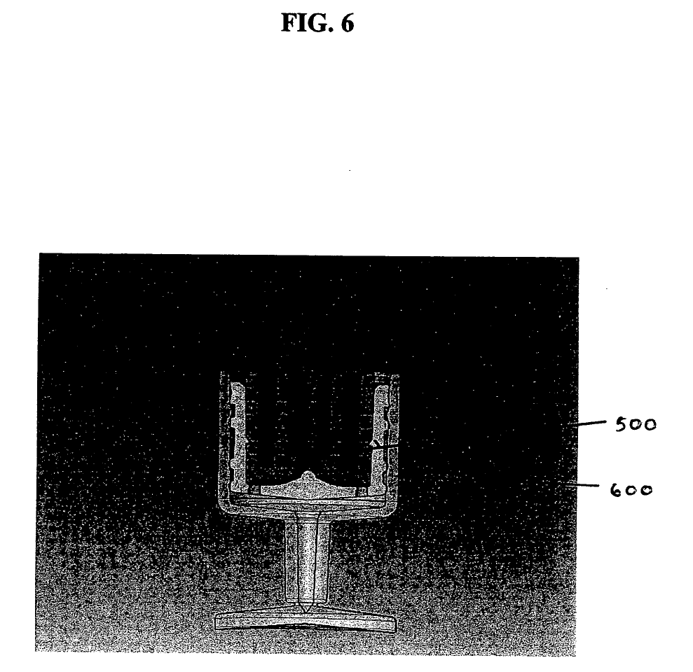

FIG. 6 shows a cross sectional area of an embodiment of the container closure assembly 600 upon completion of the freeze drying cycle whereupon the liquid active ingredient has formed into a dry powder and the plunger assembly 500 has been compressed by the freeze dryer shelves to create a sealed container closure assembly 600.

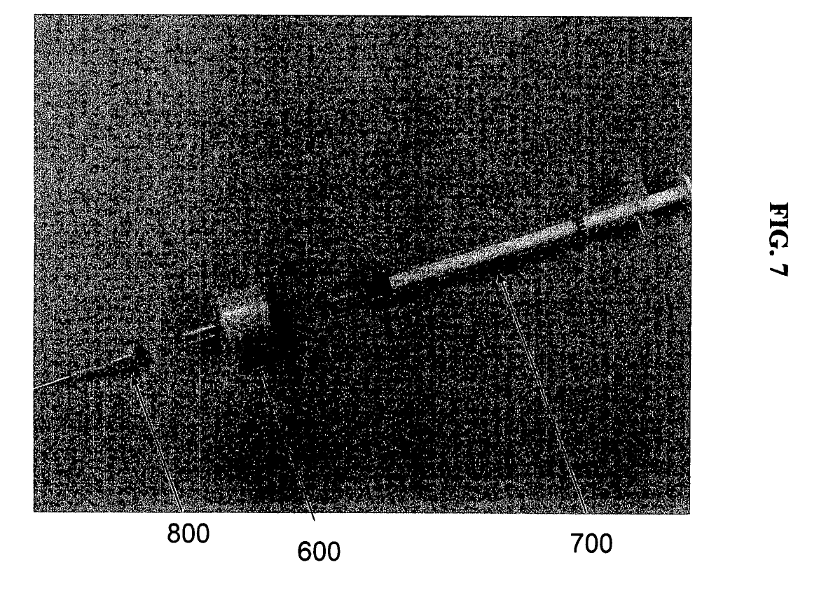

FIG. 7 shows the intended use of the sealed container closure assembly 600 of the present invention with a pre-filled syringe 700 and needle 800. In FIG. 7, the base 110 attached to the neck area 120 of the assembly 600 has been broken off to allow for attachment of a needle 800.

Contemplated for use in the container closure assembly of the present invention are storage stable powder formulations of pharmaceutical products. Importantly, the powder formulations of the present invention are optimized to produce powders which provide for “rapid” dissolution of the lyophilized powder, i.e., the powders are readily and immediately dissolved upon contact with a liquid diluent. The lyophilized powders of the present invention comprise an active ingredient, e.g., protein, and a stabilizer. Stabilizers are added to the lyophilized formulation to enhance the stability of active ingredient. Stabilizers such as, e.g., surfactants, sugars, polymers, antioxidants, amino acids, can be added to stabilize active ingredient during freezing process; and additives that can replace hydrogen bonds of water during dehydration process, e.g., sucrose, trehalose, lactose, or other sugars, can be added to stabilize pharmaceuticals by maintaining their native structure.

In order to maintain large surface area, the powder formulations may further comprise bulking agents that can form crystalline matrices (e.g., mannitol, glycine, polyethylene glycol, and the like). Alternatively, other glassy bulking agents like sugars and polymers, e.g., sucrose, trehalose, lactose, proteins, dextran and its derivatives, cyclodextran, carboxymethylcellulose, PVA, PVC, startch and its derivatives, can be added to the formulation.

The powder formulations may further comprise surfactants and buffers. Such surfactants include polysorbate 80 (or Tween 80), polysorbate 20 (or Tween 20), or pluronics. Such buffers include, e.g., phosphate, histidine, imidazole, citrace, acetate, succinate, glutamate, and glycine can be added to keep desirable pH.

In order to minimize the mass that needs to be dissolved during injection, the formulation can be composed mostly by active ingredients. For example, protein or peptide products can be lyophilized with the final solid content of 95% of protein or peptide and 5% of stabilizer.

Pharmaceutical products (active ingredients) contemplated for use include small molecules, vaccines, live or attenuated cells, oligonucleotides, DNA, peptides, and recombinant or naturally occurring proteins, whether human or animal, useful for prophylactic, therapeutic or diagnostic application. The active ingredient can be natural, synthetic, semi-synthetic or derivatives thereof. In addition, active ingredients of the present invention can be perceptible. A wide range of active ingredients are contemplated. These include but are not limited to hormones, cytokines, hematopoietic factors, growth factors, antiobesity factors, trophic factors, anti-inflammatory factors, and enzymes One skilled in the art will readily be able to adapt a desired active ingredient to the powdered formulations of present invention.

Active ingredients can include but are not limited to insulin, gastrin, prolactin, adrenocorticotropic hormone (ACTH), thyroid stimulating hormone (TSH), luteinizing hormone (LH), follicle stimulating hormone (FSH), human chorionic gonadotropin (HCG), motilin, interferons (alpha, beta, gamma), interleukins (IL-1 to IL-12), interleukin-1 receptor antagonists (IL-1ra), tumor necrosis factor (TNF), tumor necrosis factor-binding protein (TNF-bp), erythropoietin (EPO), granulocyte-colony stimulating factor (G-CSF), stem cell factor (SCF), leptin (OB protein), brain derived neurotrophic factor (BDNF), glial derived neurotrophic factor (GDNF), neurotrophic factor 3 (NT3), fibroblast growth factors (FGF), neurotrophic growth factor (NGF), bone growth factors such as osteoprotegerin (OPG), insulin-like growth factors (IGFs), macrophage colony stimulating factor (M-CSF), granulocyte macrophage colony stimulating factor (GM-CSF), megakaryocyte derived growth factor (MGDF), keratinocyte growth factor (KGF), thrombopoietin, platelet-derived growth factor (PGDF), novel erythropoiesis stimulating protein (NESP), bone morphogenetic protein (BMP), superoxide dismutase (SOD), tissue plasminogen activator (TPA), urokinase, streptokinase and kallikrein. The term proteins, as used herein, includes peptides, polypeptides, consensus molecules, analogs, derivatives or combinations thereof

In one embodiment of the present invention, the lyophilized formulation comprises a protein drug substance, interleukin-1 receptor antagonist (IL-1ra), and standard excipients, glycine, sucrose and polysorbate 20.

Diluent to be used with the powders contained within the container closure assembly can also be customized for the best stability and patient compliance. Diluents contemplated for use include commercially available water for injection (WFI), bacteriostatic water for injection (BWFI), or phosphate buffered saline (PBS), etc. Custom developed diluent can further contain a buffering agent, e.g., acetate, phosphate, histidine, citrace, acetate, succinate, glutamate, and glycine; surfactants; stabilizers; tonicity modifiers like sodium chloride; metal ions; local anesthetic agents like lidocaine or benzyl alcohol, and hydrogels for controlled release, etc.

Materials contemplated for use in the manufacturing of the product container and the hard plug portion of the present invention include, e.g., polycarbonate, polystyrene, Teflon, and the like. Such materials are well known to those of ordinary skill in the art and readily available.

Materials contemplated for use in the manufacturing of the soft plug portion of the present invention include rubber or other pharmaceutically acceptable material that offer appropriate sealing properties. Such materials are well known to those of ordinary skill in the art and readily available.

It is understood that the container closure assembly of the present invention may vary in size and is readily adaptable to and functional with any standard type pre-filled syringe and standard type needles. Such syringes and needles are well known to those of ordinary skill in the art and readily available. Generally, the container physical dimensions should be roughly no more than 15 mm×15 mm×15 mm and the container should have provisions for filling up to 1.5 ml of liquid pharmaceutical product to be lyophilized.

In the improved process for the preparation of a container closure assembly containing a lyophilized powder product, 1) the empty product container is loaded into a industry standard vial manufacturing filling line in a similar manner as regular vials; 2) the product container is filled with an optimized liquid formulation containing a pharmaceutical product; 3) the hard plug component is inserted snugly into the soft plug component to create a plunger assembly; 4) the plunger assembly is dropped into an “open” position on top of the product container, sealing the product container in the same manner as lyophilization stoppers are mounted to regular vials, creating a container closure assembly; 5) the container closure assembly is then placed into the lyophilizer and subjected to a lyophilization process; 6) during lyophilization, vapor escapes via the openings within the plunger assembly; and 7) upon completion of lyophilization, vertical compression of the lyophilizer shelves will seal the plunger assembly into the product container creating a sealed container closure assembly with minimal head space and which retains the sterility of the pharmaceutical product. Importantly, in this process, the plunger assembly is compressed such that it rests directly on top of the pharmaceutical powder and there is no air space between the powder and the plunger assembly (see FIG. 6). This design concept facilitates the direct injection of the lyophilized powder without the need for a separate reconstitution/mixing/priming step of powder with diluent. In addition, the sealed container closure assembly of the present invention is able to retain the sterility of the pharmaceutical powder product and is storage stable at room temperature over the shelf life of the product.

In the improved method for the administration of a lyophilized pharmaceutical product using the container closure assembly of the present invention, 1) the sealed container closure assembly is attached at one end via friction fit to either a luer-lock or luer-slip pre-filled syringe containing the diluent; 2) the detachable base located on the neck end of the container closure assembly is removed by applying a tangential force at the base, thus exposing a luer-slip tip for the attachment of a needle; 3) a luer-slip needle is attached via friction fit to the exposed luer-slip tip of the container closure assembly; 4) the injection is then initiated by inserting the needle into the injection site; and 5) force is applied to the syringe plunger whereupon the diluent in the syringe will be forced through the plunger assembly (more specifically, the diluent will flow through Component A and into Component B via the one-way valve created by the union of Components A and B, then flow through the central channel and exit the openings in Component B); 6) the diluent will encounter the lyophilized powder in Component C and rapidly reconstitute; and 7) the reconstituted liquefied product mixture exits the container closure assembly at the luer-tip at the end of the neck area of Component C, passes through the attached needle and into the injection site. As an alternative to steps 2) and 3), the container closure assembly may have a staked needle (with a needle shield) attached at the neck end, and the needle shield removed prior to performing step 4). Importantly, the method does not require a separate reconstitution/mixing/priming step, thereby providing for a more convenient and ease of use for the patient and/or end user.

And, importantly, the improved delivery method of the present invention provides a ‘gradient delivery’ of the injectable pharmaceutical product. For example, because the present invention provides for the immediate reconstitution of the powdered drug upon contact with the diluent, the product is injected into the patient in a manner wherein more highly concentrated product is injected initially. It is the improved process and container closure assembly design concept described herein that facilitates the direct administration of the powdered active ingredient, without the need for a separate reconstitution/mixing step. It is thus envisioned that the lyophilized formulations, lyophilization processes and closure assembly design concepts described herein could be applied to existing delivery devices, e.g., pen systems, autoinjector systems, needle-free injector systems, dual-chambered injection cartridges and/or pre-filled syringe systems, to provide for improved methods of administration which provide for gradient delivery and which are more user friendly for the patient and/or end user.

### EXAMPLE 1

In this Example, a study was conducted to demonstrate the ‘gradient delivery’ injection profile associated with the administration of a powdered drug using the formulations, lyophilization processes and container closure assembly design of the present invention.

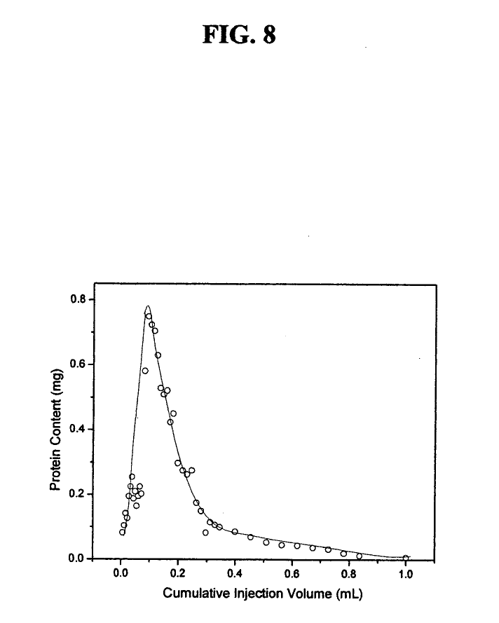

The study was performed utilizing a model protein drug substance, interleukin-1 receptor antagonist with standard excipients glycine, sucrose and polysorbate 20. The study was performed by using a sealed container closure assembly prepared using the process of the present invention and containing 10 mg of IL-1ra powder which was dried in a typical lyophilization process. A syringe containing 1 ml of diluent (water) was attached to the plunger assembly of the container closure assembly and the detachable base at the neck end of the container closure assembly was removed. Force is applied to the syringe plunger such that the water flows through the assembly, reconstitutes the powder, and the resultant solution drips out of the ejection port of the assembly. The concentration of IL-1ra in each drop of solution was measured with a ultraviolet spectrometer. The data collected and shown in FIG. 8 characterize the general profile of the gradient delivery associated with the administration of a powdered drug using the formulations, lyophilization processes and container closure assembly design of the present invention. As depicted in FIG. 8, the concentration of the dose delivered over the injection volume for a gradient delivery is non constant with the bulk of the active pharmaceutical ingredient being delivered during the initial portion of the injection.

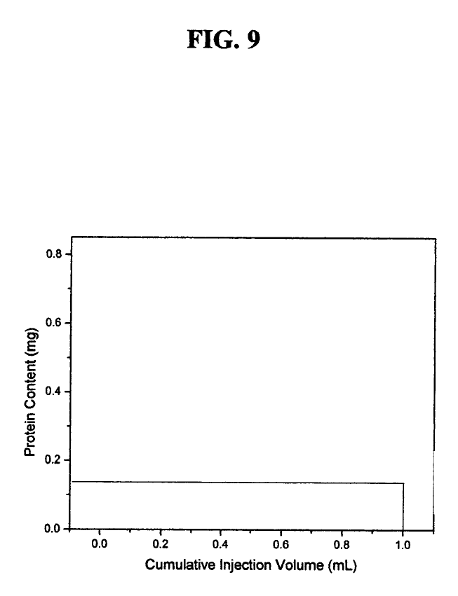

This unique gradient delivery of the injectable pharmaceutical powder product may be advantageous to the patient in certain therapeutic settings. To date, none of the known prior art delivery techniques and devices used for delivery of powdered drugs have such a profile, as all require a reconstitution and/or mixing step of the powdered drug with a diluent prior to injection, and therefore have an injection profile similar to that depicted in FIG. 9. Although this specific protein was used, it is highly probable that for those skilled in the art and for most standard active pharmaceutical products, excipients and other ingredients that the same results can be achieved and will reflect these same characteristics and injection response.

The improved lyophilized formulations, lyophilization processes and closure assembly design concepts of the present invention provide patients and end-users with an alternative, less expensive and easier to use device than current state-of-the-art delivery systems for lyophilized products. Utilization of the design concept described for container closure assembly of the present invention on existing delivery devices would provide a valuable and much needed benefit to those patients dependent upon powdered drugs in their therapeutic settings.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A delivery device for the administration of a powdered pharmaceutical product comprising:
a first component having an ejection port at one end, a chamber holding a reservoir of diluent, and an activating means to facilitate the flow of said diluent through said ejection port;
a second component containing a lyophilized drug powder with minimal head space, said second component capable of engaging said first component at one end and engaging a standard type needle at the opposing end, said second component comprising a valve mechanism which upon activation of said first component, allows the diluent ejected from said first component to encounter the powder in said second component, rapidly reconstitute and pass through said needle.

2. A device according to claim 1 wherein said second component is a container closure assembly comprising:
a hard plug component comprising:
a female luer slip fitting cavity to allow for friction fit of a standard type luer slip syringe;
a circular cavity that can accommodate a typical luer lock syringe; and
a depression and female portion that can be mated with the sealing conical mound of the soft plug component to create a one way valve;
a soft plug component comprising:
a hollow inside capable of accepting said hard plug component to create a plunger assembly;
vent holes which allow for vapors to escape during lyophilization processes;
sealing ridges which serve to seal said soft plug component against the interior wall of the product container; and
a sealing conical mound that can be mated with said female portion of said hard plug component to create a one way valve;
a product container component comprising:
an open end having a chamber capable of holding a liquid to be lyophilized and capable of receiving said plunger assembly; and
an opposing neck end having a detachable base to allow for attachment of a standard type luer slip or luer lock syringe needle.

3. A container closure assembly suitable for lyophilized pharmaceutical products comprising:
a hard plug component comprising:
a female luer slip fitting cavity to allow for friction fit of a standard type luer slip syringe;
a circular cavity that can accommodate a typical luer lock syringe; and
a depression and female portion that can be mated with the sealing conical mound of the soft plug component to create a one way valve;
a soft plug component comprising:
a hollow inside capable of accepting said hard plug component to create a plunger assembly;
vent holes which allow for vapors to escape during lyophilization processes;
sealing ridges which serve to seal said soft plug component against the interior wall of the product container; and
a sealing conical mound that can be mated with said female portion of said hard plug component to create a one way valve;
a product container component comprising:
an open end having a chamber capable of holding a liquid to be lyophilized and capable of receiving said plunger assembly; and
an opposing neck end having a detachable base to allow for a friction fit of a standard type luer slip or luer lock syringe needle.

4. An improved process for the preparation of a container closure assembly containing a lyophilized pharmaceutical powder product comprising the steps of:
1) loading an empty product container into an industry standard vial manufacturing filling line;
2) filling said product container with a liquid formulation containing a pharmaceutical product;
3) dropping a plunger assembly, the plunger assembly comprising a hard plug component inserted into a soft plug component, into the top of said product container to create a container closure assembly;
4) placing said container closure assembly into a lyophilizer apparatus;
5) subjecting said container closure assembly to a lyophilization process; and
6) completing said lyophilization process such that vertical compression of the lyophilizer shelves seals said plunger assembly into said product container to create a sealed container closure assembly containing lyophilized pharmaceutical powder product with minimal head space.

5. An improved method for the administration of a lyophilized pharmaceutical powder product comprising the steps of:
1) providing a delivery device, said device having a first component and second component, said first component having an ejection port at one end, a chamber holding a reservoir of diluent, and an activating means to facilitate the flow of said diluent through said ejection port; said second component containing a lyophilized drug powder with minimal head space and capable of engaging said first component at one end and engaging a standard type needle at the opposing ejection port, said second component comprising a valve mechanism which upon activation of said first component, allows the diluent ejected from said first component to encounter the powder in said second component and flow through the ejection port of said second component;
2) attaching a standard type needle to the ejection port of said second component;
3) initiating an injection by inserting said needle into the injection site; and
4) activating said first component whereupon the diluent will encounter the lyophilized powder in said second component and rapidly reconstitute and pass through said needle into the injection site.

6. A method to provide for the gradient delivery of a lyophilized pharmaceutical powder product comprising the steps of:
1) providing a delivery device, said device having a first component and second component, said first component having an ejection port at one end, a chamber holding a reservoir of diluent, and an activating means to facilitate the flow of said diluent through said ejection port; said second component containing a lyophilized drug powder with minimal head space and capable of engaging said first component at one end and engaging a standard type needle at the opposing ejection port, said second component comprising a valve mechanism which, upon activation of said first component, allows the diluent ejected from said first component to encounter the powder in said second component and flow through the ejection port of said second component;
2) attaching a standard type needle to the ejection port of said second component;
3) initiating an injection by inserting said needle into the injection site; and
4) activating said first component whereupon the diluent will encounter the lyophilized powder in said second component and rapidly reconstitute and pass through said needle into the injection site.

7. An improved method for the administration of a lyophilized pharmaceutical powder product comprising the steps of:
1) providing a sealed container closure assembly, the assembly containing a lyophilized pharmaceutical powder product with minimal head space;
2) attaching a standard type syringe, the syringe containing a diluent, to one end of said sealed container closure assembly;
3) attaching a standard type needle to the opposing end of said sealed container closure assembly;
4) initiating an injection by inserting said needle into the injection site; and
5) applying force to said syringe plunger whereupon the diluent in said syringe will be forced through said sealed container closure assembly, encounter the lyophilized powder and rapidly reconstitute, exit said container closure assembly and pass through said needle into the injection site.

8. A method to provide for the gradient delivery of a lyophilized pharmaceutical powder product comprising the steps of:
1) providing a sealed container closure assembly, the assembly containing a lyophilized pharmaceutical powder product with minimal head space;
2) attaching a standard type syringe, the syringe containing a diluent, to one end of said sealed container closure assembly;
3) attaching a standard type needle to the opposing end of said sealed container closure assembly;
4) initiating an injection by inserting said needle into the injection site; and
5) applying force to said syringe plunger whereupon the diluent in said syringe will be forced through said sealed container closure assembly, encounter the lyophilized powder and rapidly reconstitute, exit said container closure assembly and pass through said needle into the injection site.

9. A container closure assembly suitable for lyophilized pharmaceutical product comprising:
a tubular body having an ejection port at one end, said ejection port having a means to allow for attachment of a standard type syringe needle; a plunger axially slidable in the body; a first chamber holding a reservoir of diluent; a second chamber containing a lyophilized drug powder with minimal head space; and a by-pass mechanism formed between said first and second chambers;
wherein upon the pressing of said plunger, said by-pass mechanism allows the diluent from said first chamber to encounter the powder in said second chamber, rapidly reconstitute, and pass through said attached needle without the need for a separate reconstitution/mixing/priming step.
